(19) United States
(12) Patent Application Publication
(10) Pub. No.: US 2015/0374897 A1
(43) Pub. Date: Dec. 31, 2015

(54) APPARATUS FOR BLOOD PURIFICATION BY EXTRACORPOREAL CIRCULATION

(71) Applicant: INFOMED SA, ACACIAS (CH)

(72) Inventor: Olivier FAVRE, Thonex (CH)

(21) Appl. No.: 14/746,088

(22) Filed: Jun. 22, 2015

(30) Foreign Application Priority Data
Jun. 26, 2014 (EP) 14174461.5

(51) Int. Cl. A61M 1/36

(52) U.S. Cl. CPC A61M 1/3663

(57) ABSTRACT

A device for purifying blood includes an extracorporeal blood circulation loop, flow rate control elements to manage the blood circulation in the extracorporeal blood circulation loop, blood purification elements configured on the extracorporeal blood circulation loop, citrate injection elements for injecting a solution including citrate into the extracorporeal blood circulation loop, and a control unit configured to control the citrate injection elements and the flow rate control elements. The control unit is further configured to require, at determined time intervals of a blood purification treatment used by the device, the entry of at least one representative value of the status of the device and/or of the patient. The control unit then verifies the adequacy of the representative value with pre-established rules stored in the control unit and controls the citrate injection elements and/or the flow rate control elements in accordance with the result of the verification and the pre-established rules.

### FIELD OF THE INVENTION

This invention relates to a device for extracorporeal purification of blood.

### BACKGROUND OF THE INVENTION

Several methods for purifying blood exist that are applied as a function of criteria described in the literature for more than 40 years. In particular, hemodialysis, hemofiltration, hemodiafiltration, plasma exchange, double filtration or the adsorption by blood or by plasma extracted from blood can be cited. These treatment means, generally performed by a veno-venous approach (the blood is taken from and returned into a vein), are extensively described in the literature and can, in the most serious clinical cases, be performed continuously (24 hours a day). They are found now under the names: CVVHD (continuous veno-venous hemodialysis), CVVH (continuous veno-venous hemofiltration), and CVVHDF (continuous veno-venous hemodiafiltration).

All of these techniques, to which the device according to this invention can be applied, have in common the need for extracorporeal blood circulation, i.e., a blood circulation loop external to the patients that includes blood circulation means and one or more elements that make possible the exchange with the blood to purify the latter of substances associated with disease. Because of the coagulating properties of blood, an anticoagulant is often added to the blood upstream from the loop, before the exchange element. The two most customary products to prevent coagulation of blood are heparin and citrate.

Heparin, which acts by activating antithrombin III, is generally used for systemic anticoagulation, that is to say that not only the blood is anticoagulated in the circulation loop but it remains so when it returns to the patient, which leads to, among other things, the risk of bleeding.

Citrate binds itself to calcium, which is an unavoidable element of the coagulation phenomenon. It has been demonstrated that a concentration of ionized calcium (Ca⁺⁺) in the blood that is less than 0.4 mmol/L of blood increases the coagulation time, the curve having an exponential form and the blood no longer coagulating from 0.2 mmol/l (Kutsogiannis et al., Regional Citrate Anticoagulation in Continuous Veinoveinous Hemodiafiltration, American Journal of Kidney Diseases, Vol. 35, No. 5 (May), 2000: pp. 802:811; Calatzis et al., Citrate Anticoagulation for Extracorporeal Circuits: Effects on Whole Blood Coagulation Activation and Clot Formation, Nephron 2001; 89: 233-236). In clinical practice, it has been demonstrated that reducing the ionized calcium Ca⁺⁺ below 0.5 mmol/l makes it possible to extend the life of the extracorporeal circulation circuit (Nurmohamed et al., Continuous Veinoveinous Hemofiltration with or without Predilution Regional Citrate Anticoagulation: A Prospective Study, Blood Purification 2007; 25: 316-323).

The calcium bound to the citrate is in part eliminated by all of the techniques of filtration and dialysis cited previously, as well as the calcium that remains ionized. Therefore, it is necessary to inject some calcium into the patient so as to compensate for these losses and to maintain a “normal” calcium level of the patient, of 1.1 to 1.3 mmol/L, to protect him from hypocalcemia. This is done most often on the return line of the blood and makes it possible to inject into the patient a blood having an ionized calcium that is close to the one extracted. Regional coagulation is then spoken of since it is limited to the extracorporeal circulation loop, which makes it possible, particularly relative to heparin, to reduce the risk of bleeding for the patient (Park et al., Regional anticoagulation with citrate is superior to systemic anticoagulation with heparin in critically ill patients undergoing continuous veinoveinous hemodiafiltration, original article, DOI: 10.3904/kjim 2011.26.1.68) and to extend the service life of the extracorporeal circulation circuit (Sheldon et al., A Novel Regional Citrate Anticoagulation Protocol for CRRT Using Only Commercially Available Solutions, Journal of Critical Care, Vol. 18, No. 2 (June), 2003: pp. 121-129).

It should be noted that a complete model of anticoagulation by injection of citrate must also include the bonds of magnesium with citrate, the exchanges between the ionized calcium Ca⁺⁺ and the calcium bonded to the albumin, the concentration of platelets, the hematocrit count, the coagulation time, the pH variations of the patient or even the half-life of the citrate in the body of the patient. Models have been developed for specific cases, for example (Kozik-Jaromin, Citrate Kinetics during Regional Citrate Anticoagulation in Extracorporeal Organ Replacement Therapy, Aus des Medizinischen Universitätklinik Abteilung Innere Medizin IV (Nephrologie˜1 Allgemeinmedizin) der Albert-Ludwigs-Universität Freiburg i. Br., 2005) for chronic hemodialysis of a standard period of four hours, to evaluate a priori the doses of citrate and of calcium necessary for the regional anticoagulation of the blood. The patent US 2011/0237996 itself describes a device that uses mathematical models to determine a priori the necessary values and during the treatment a correction based on statistical values of parathyroid hormone and of alkaline phosphatases. Such a device apparently makes it possible to treat patients suffering from chronic kidney failure, therefore stable patients who come three times a week to the hospital and whose clinical situation is well known by the health care providers.

However, these models would often result in significant deviations of concentrations of the solutes in the cases of continuous treatments, a reason for which patents specific to these treatments exist. The patent US 2011/0168614 can be cited, which patent mentions the equations necessary to define the inputs and outputs of citrate and calcium mainly, but also of other parameters that can affect their equilibrium, for all of the continuous-type treatments (CVVHD, CVVH, CVVHDF) while being based particularly on the concentrations and the flow rates. The authors claim to be able, on the basis of their circulation model of the extracorporeal fluids, to control the calcium pump used to restore the calcium level of the patient before returning his blood to him. The problem here is that what happens in the patient is not taken into account, particularly and by way of example the metabolism of the injected citrate. The patent US 2011/0288464 finds a solution for this in part by proposing controlling the citrate and/or calcium pumps based, on the one hand, on a mathematical model comparable to that of the patent US 2011/0168614 and, on the other hand, by adding to it automatic measurements of parameters such as the flow rate of the blood or the concentration of calcium in the circuit. If such a device is a step in the right direction, it does not include the clinical circumstances of the patient since those are not reflected solely by measurable values in the blood but also by other parameters such as, for example, his cardiac, inflammatory or respiratory circumstances. The choice of flow rates must therefore be the responsibility of the health care providers and cannot be entrusted solely to the device on the basis of the values measured by the latter and of rules of computation. This is true in all of the cases of extracorporeal circulation, but it is much more so for patients undergoing continuous treatments because of their serious and complex clinical circumstances but also because of the fact that these circumstances change during the treatment.

In practice, the numerous treatment protocols that use a citrate anticoagulation have, on the one hand, checks at specific intervals of time and, on the other hand, step-by-step corrections as functions of measured values and targeted values provided by the mathematical model used. By way of example, one can see Tables 2 and 3 of the document Amanzadeh et al., Anticoagulation and Continuous Renal Replacement Therapy, Seminar in Dialysis, Vol. 19, No. 4 (July-August) 2006, pp. 311-316 that indicate for the first table the recommendations for modifying the citrate flow rate as a function of the ionized calcium Ca⁺⁺ measured after the filter (post-filter) and for the second table those applicable to the calcium flow rate as a function of the ionized calcium of the patient. In the two cases, for each measured value range, a recommended variation of flow rate is indicated with a time limit before the next check and potentially the next adjustment. For example, in Table 2, if the Ca⁺⁺ is between 0.41 and 0.45, 20 ml/h is added to the current flow rate of citrate, and after one hour, the following verification is made. In another example, Table 3 indicates that if the ionized calcium is beyond 1.35 mmol/L, the current flow rate of calcium must be reduced by 1.1 mmol/h, the next check having to be made in four hours.

These intervals of time can be evaluated as a function of the present situation and of the modifications under consideration. Actually, the closer the situation is to the one that is desired, the less significant is the possible modification and the greater the time interval before the next check because the risk for the patient is low. Conversely, if one finds oneself in a situation that is far from the one that is desired, the time interval before the next check will be short because the correction and the risk for the patient will be significant. In addition to the risk for the patient and in addition to the magnitude of the required modifications, the time interval incorporates the time to balance the system that depends in particular on the half-life of the citrate, which varies from thirty minutes to two hours as a function in particular of the hepatic situation of the patient.

The preceding examples show that the corrections of flow rates occur gradually as a function of the separation between the measured parameter and its targeted value and that the result of these corrections can be verified only after a time that is measured in terms of an hour or a time during which the health care providers will perform a number of other tasks. A deadline can thus easily be allowed to pass which gives rise to risks intrinsic to the citrate anticoagulation systems with a compensation of calcium. These risks come directly from four possible cases that are: too much or too little of citrate and too much or too little of calcium. Some of these risks can bring about a significant degradation of the clinical status of the patient, and even his death; they are extensively described in the literature (see Davenport et al., Citrate Anticoagulation for Continuous Renal Replacement Therapy (CRRT) in Patients with Acute Kidney Injury Admitted to the Intensive Care Unit, NDT Plus (2009) 2: 439-447) and often cited as a major limitation to the use of citrate as anticoagulant. It therefore appears significant to limit them as much as possible, but the devices for blood purification by extracorporeal circulation that use citrate as anticoagulant, as described in the prior art, are not inherently safe because they do not require any validation of the adequacy of the anticoagulation by the health care providers nor do they check, at adequate time intervals, treatment values entered by said providers as a function of established rules and of automatically measured values, for example the ionized calcium. In addition, they do not incorporate the maximum tolerance of the patients to citrate, nor the time spent in the circuit as a function of its volume and of the blood flow rate, nor the variations coming from the metabolism of the citrate by the patient, three parameters that can have a major influence on the risks incurred by the patient.

The purpose of this invention is to eliminate, at least in part, the above-mentioned drawbacks and to produce a device for purifying blood by extracorporeal circulation with citrate anticoagulation, which is safe and suitable for use in continuous treatment.

### SUMMARY OF THE INVENTION

This invention therefore has as its object a device that makes it possible to achieve a citrate anticoagulation that, to be safe, requires the knowledge at validated intervals of time of at least one representative value of the circumstances of the anticoagulation and that uses this value to authorize, or reject, the variations in flow rates of treatments (blood, dialysis, substitution, loss of weight, citrate, calcium). The time interval can be variable, for example shorter at the beginning of treatment or after one or more parameters have been modified so as to better cover the periods of stabilization and to reduce the risks for the patient and longer under stable conditions, i.e., when the system does not undergo variations particularly when the representative value is near the one that is desired, so as to save time and money by avoiding unnecessary measurements. This also involves, in the same way as a well-managed anticoagulation, reducing the blood losses of the patient as well as the associated risks of transfusion.

### BRIEF DESCRIPTION OF THE DRAWINGS

The accompanying drawings show diagrammatically and by way of example an embodiment of a device for purifying blood by extracorporeal circulation according to the invention.

FIG. 1 shows diagrammatically a device according to the invention.

FIG. 2 shows diagrammatically the control unit of a device according to the invention.

FIG. 3 shows diagrammatically the operation of the device according to the invention.

### DETAILED DESCRIPTION OF THE INVENTION

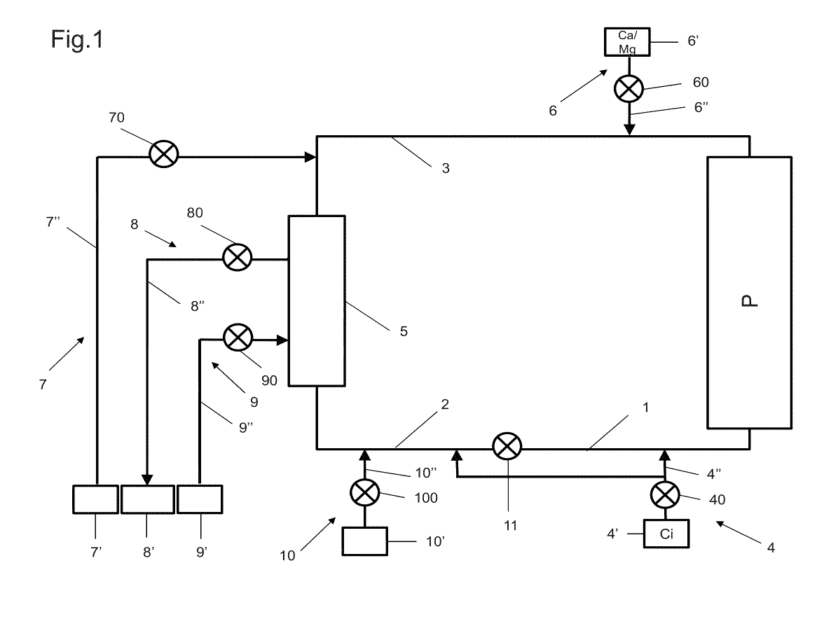

A purification device according to the invention has, as shown in FIG. 1, an extracorporeal blood circulation loop comprising a removal line 1, means for circulation and for control of blood flow rate 11, such as a pump, to remove the blood from the body of the patient P, a return line 3 to bring the purified blood back into the body of the patient P, and blood purification means 5 that are placed between an intermediate line 2 connected to the blood circulation means 11 downstream from the latter and the return line 3 and that consist of, for example, filters, dialyzers or adsorption cartridges.

Means 4 making it possible to inject a fluid containing citrate (Ci) are placed on the blood circulation loop 1, 11, 2, 3. These means 4 comprise a bag 4′ containing the fluid to be injected, a line 4″ connecting the bag 4′ to the removal line 1 or to the intermediate line 2, and a pump 40 making it possible to inject the contents of the bag 4′ into the removal line 1 or the intermediate line 2 by way of the line 4″.

Optionally, the device can be completed by means 6 making it possible to inject calcium (Ca) and/or magnesium (Mg) (either by a single element as shown or by separate elements—one for calcium and the other for magnesium) into the return line 3, and by treatment means 7, 8, 9, 10 making it possible to perform the known purification techniques. The means for injecting the calcium/magnesium 6 can comprise in particular a line 6″ connecting a bag 6′ containing a solution comprising calcium and/or magnesium to the removal line 3 and a pump 60 for the injection of the contents of the bag into the circulation loop. In FIG. 1, the reference 9 designates in particular means for injecting a dialyzate comprising a dedicated line 9″ connected to the blood purification means 5, a dialyzate bag 9′ and a pump 90; the reference 8 designates means for removing the contaminated solution comprising in particular a removal line 8″ connected to the blood purification means 5, a bag for the contaminated liquid 8′ and a removal pump 80; the reference 7 designates means for injecting post-dilution replacement liquid comprising a post-dilution line 7″ connected to the removal line 3, a post-dilution pump 70, and a post-dilution bag 7; and the reference 10 designates means for injecting a pre-dilution liquid comprising a line 10″ connected to the intermediate line 2, for example, a pre-dilution bag 10′ and a pre-dilution pump 100. The dialyzate injection means 9 and the contaminated solution removal means 8 make it possible in particular to perform a hemodialysis. The pre-dilution means 10, the contaminated solution removal means 8, and the post-dilution means 7 make it possible to perform a hemofiltration, and the dialyzate injection means 9, the contaminated solution removal means 8, the post-dilution means 7 and/or the pre-dilution means 10 make it possible to perform a hemodiafiltration.

The blood loop 1, 2, 3 can be completed in practice by the means that are customary but not shown for protection of the circuit, such as, for example, pressure sensors, or for protection of the patient, such as, for example, a blood leak detector or an air detector associated with a clamp. These means are widely known to a person skilled in the art and are described in the literature.

Means for circulation and for flow rate control other than the pumps 11, 40, 100, 90, 80, 70 and 60 mentioned above exist (for example, clamps) and are described in the literature and can obviously be used in this invention instead of said pumps.

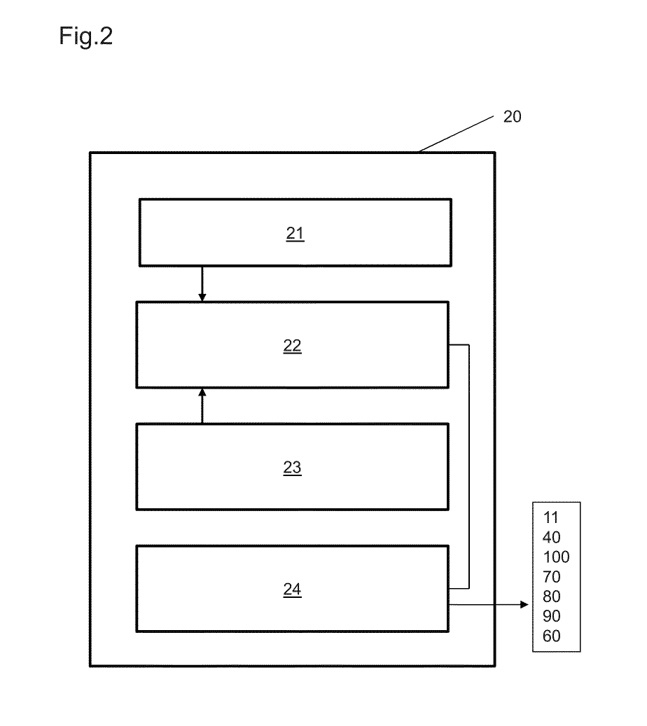

The device according to the invention has in addition a control unit 20 shown in FIG. 2 and comprising in particular a user interface 21, a calculating unit 22, a memory 23, and control means 24 connected to each of the means for circulation and for flow rate 11, 40, 60, 70, 80, 90, 100 present on the blood circulation loop 1, 2, 3, 4 and making it possible to control said means for circulation and for flow rate.

According to the invention, the control unit 20 is programmed to force the user to insert, by way of the user interface 21, at least one established parameter as being a representative value and that makes possible the checking of the adequacy of the treatment and anticoagulation values, at a time interval that is determined according to pre-established rules and placed in the memory 23.

According to the models used for the treatment, the representative value can be, for example, the level of ionized calcium at a point of the blood circulation loop 1, 2, 3, 4, the pH of the patient or his total calcium, and all other values that are known to be representative in the model under consideration. Hereinafter, the expression “representative value” designates any one of these values and/or any combination or set of these values. The user interface 21 also makes possible the entry by the user of the treatment and anticoagulation values and the display of messages necessary for the proper implementation of the treatment.

The calculating unit 22 verifies the required treatment and anticoagulation parameters, i.e., the flow rates of the circulation means 40, 60, 70, 80, 90, 100, 11, or of some of them, as a function of the pre-established rules and of the representative value previously entered by the user, and sends to the user interface the messages necessary for updating the treatment. If the required values are acceptable for the calculating unit 22, it then sends the corresponding orders to the control means 24 of the fluid circulation means 40, 60, 70, 80, 90, 100, 11, or some of them.

The pre-established rules rely on known equations but can also incorporate new conditions, particularly high and low limits. Among the known equations, there can particularly be cited the fact that to each fluid, a flow rate “Q” corresponds that is represented by way of example by the pump(s) 40, 60, 70, 80, 90, 100, 11. For each substance contained in a fluid, the amount injected “M” in terms of unit of time “t” is provided by the product of the concentration “C” with the corresponding flow rate or: M=Q*C. Since the flow rates and the concentrations are known by the device, either because they are entered by way of the user interface 21 or because they are automatically calculated by the calculating unit 22, it is possible to calculate the masses of the substances that interest us, particularly those of the citrate and of the calcium, and to deduce from them acceptance values by applying additional rules such as, for example, the balance between calcium that is injected and lost in the circuit or the maximum admissible citrate mass. The flow rate and concentration values that must be calculated are in accordance with the rules known to a person skilled in the art.

To illustrate this with the example of a hemofiltration treatment, the operator supplies a post-dilution substitution flow rate used to control the pump 70 and a weight loss flow rate; the calculated discharge flow rate of the pump 80 is then equal to the sum of the substitution flow rate and of the weight loss so as to remove weight (in this particular case mainly in the form of water) from the patient in accordance with the instruction of the operator.

Another example would be the case of the calculation of the concentration of citrate in the blood that is circulating in the purification means 5 (Cci₅), considering that the pre-dilution pump 100 is stopped and therefore does not affect the calculation. In this case, the concentration would be calculated from that in the bag 4′ (CCi₄), of fluid containing citrate, and flow rates of citrate QCi and of blood Qb with: Cci₅=CCi₄*(QCi/(Qb+QCi)). Thus, for each point of the circulation loop 1, 2, 3 shown in FIG. 1, the flow rates and concentrations of the substances that interest us can be known, either because they are entered by way of the interface 21 by the operator or by calculation. The detailed calculations that can be applied to this invention in their complete form or another simplified form have already been the object of publications (see Kozik-Jaromin, Citrate Kinetics during Regional Citrate Anticoagulation in Extracorporeal Organ Replacement Therapy, Aus des Medizinischen Universitätklinik Abteilung Innere Medizin IV (Nephrologie˜1 Allgemeinmedizin) der Albert-Ludwigs-Universität Freiburg i. Br., 2005) and patents US 2011/0288464 and US 2011/0168614.

To make the treatment safer, other rules must also be applied, particularly those that define the limit values of citrate. Actually, on the one hand, the patient cannot tolerate without unwanted secondary effects an injection of citrate beyond an established value, at 80 mg/kg/h in the literature, or involving a pH greater than 7.45 and, on the other hand, a value that is too low that would increase the risk of coagulation in the loop. Because of an incomplete model that means that since the citrate is bonding to the calcium and to the magnesium, the more blood there is, the more calcium and magnesium there is with which to bind, and therefore the more citrate is necessary to maintain the targeted value of calcium and the coagulation time, the flow rate of citrate is often defined as a ratio of the blood flow rate. The problem at a high blood flow rate is that too much citrate is injected relative to what the patient can tolerate and to what is useful since, with the time in the circuit diminishing when the blood flow rate increases, the anticoagulation can be less at a high blood flow rate. Conversely, at a low blood flow rate, the time in the circuit is long and at a proportional citrate flow rate, therefore constant anticoagulation, the risk of coagulation in the loop increases even though the injected citrate is far below the acceptable limit value for the patient. In this case, there would therefore be interest in injecting proportionally more citrate into the blood so as to increase the coagulation time. Thus, the device according to the invention can incorporate rules and values that restrict the mass of citrate injected, upward as a function of the tolerance of the patient and downward as a function of the coagulation time that is necessary to ensure that the blood travels through the circulation loop 1, 2, 3.

To the rules of evaluation described above, a device according to the invention adds rules for acceptance of the parameters entered by the operator by way of the interface 21. These rules of acceptance can depend or not upon the representative parameter of the anticoagulation. For example, a high citrate ratio with a high blood flow rate resulting in an injection known to be dangerous for the patient can be rejected, an upper limit of a citrate ratio then being able to be proposed. Another example is that if the representative parameter is, for example, the ionized calcium of the patient, and the value entered shows a value known to be low, for example less than 1.1 mmol/L, the device can reject that the injection of calcium by the pump 60 be reduced, or not accept it until after a double validation by the operator. The rules of acceptance can contain, as a function of the circumstances, one or more optimal value(s) and an associated margin of tolerance. It is quickly understood that the possibility exists of creating numerous rules of acceptance and that these are called upon to change with the knowledge of the doctor. A device according to the invention can thus use any rule aiming to reduce the risks described previously for the patient.

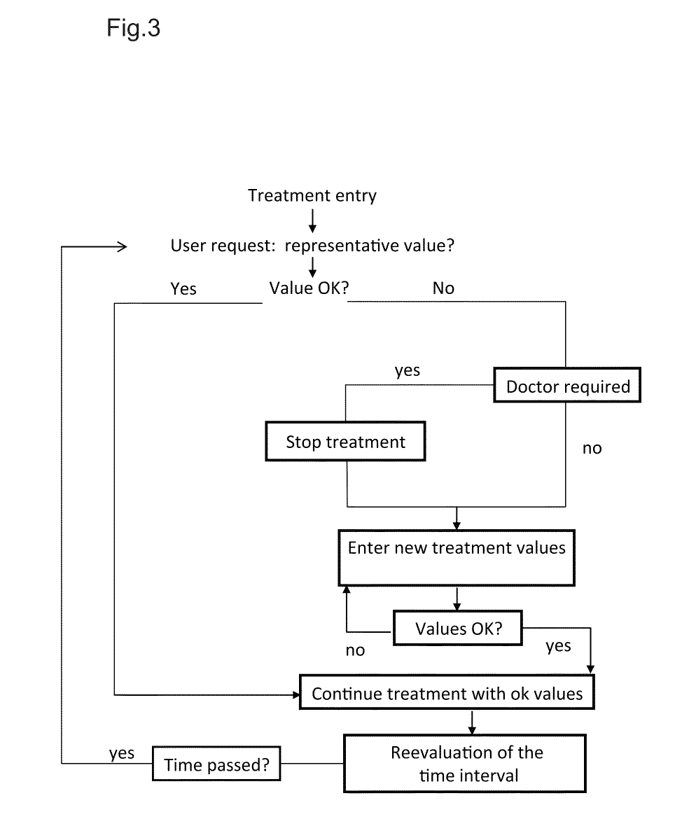

The device according to the invention operates overall as shown by way of example in FIG. 3, knowing that numerous algorithm variants meeting the same need are possible. The device asks the operator by way of the user interface 21 to enter the treatment data and the representative value(s) of the treatment at a given time. The calculating unit then uses the applicable rules and values placed in the memory 23 to verify if the data entered are acceptable. If this is the case, the calculating unit 22 sends the information to the means for controlling the pumps 24 that then feed the latter to produce the flow rates that are requested or calculated. If this is not the case, the calculating unit 22 verifies first whether the deviation is at this point significant enough that a doctor must be called, in which case it can inform the user interface 21 of it which will display a message indicating the necessity of a check by the doctor and will stop the treatment if it is underway, that is to say that it will indicate to the control means 24 to send a zero instruction to the pumps 70, 80, 90, 100 and depending on the cases 40 and/or 60. Otherwise, the device requests that the values of treatments be adjusted while respecting the applicable rules of acceptance. When the treatment values have been adjusted and are acceptable, the treatment can continue, the next time interval for the following check being reevaluated. Once this has elapsed, the device again requests entering the representative value(s) and recommences its acceptance cycle.

The reevaluation of the time interval is necessary, on the one hand, to ensure the safety of the patient and, on the other hand, to minimize the number of measurements of the representative values that necessitate time, funding, and most often blood samples. It is defined by rules and reference values stored in the memory 23. For example, at the beginning of the treatment, the interval is equal to a half-hour the first hour, then one hour for the following hour, then every four hours until twenty-four hours of treatment, then every twelve hours. This set program can be modulated, for example after a significant change defined as being a variation of at least 30% of one of the treatment parameters; it is conceivable to come back to a time interval of one hour before going again to four, then to twelve hours. In the same way, this time interval can also be modified as a function of the values received with—for example if the calcium measured is beyond a known limit that would be used to warn the doctor—a return to two measurements made every half-hour before continuing with intervals at one, four and twelve hours. The values supplied above are by way of illustration of the remarks, and the device according to the invention makes it possible as a variant that they can be modified, for example, as a technical parameter adjusted to the delivery of the device as a function of the requirements of the center that will use the device, or as parameters linked to the definition of the treatment or as parameters modified by the operator during treatment to lengthen or shorten the predetermined interval, as a function of his own knowledge.

The values entered at these intervals determined by the device are used by the acceptance rules of the treatment values. As entered value(s), it is possible to consider, by way of example, the ionized calcium of the patient, its ratio with the total calcium, the ionized calcium as input and/or output of the purification means 5 or else the pH of the patient. By incorporating these values into the calculation rules, the calculating unit 22 can then determine, on the one hand, if the treatment parameters are within acceptable ranges and, on the other hand, the time interval for the next check as well as the messages to be displayed. The device thus makes it possible to warn of the entry of treatment values that can lead to imbalances and therefore risks for the patient or of coagulation of the blood in the circulation loop 1, 2, 3.

An essential advantage of this invention lies in the fact that it makes it possible, relative to existing devices, to significantly reduce the risks connected to the use of citrate as an anticoagulant, the operator having the obligation at determined intervals of time to insert one or more determining values for the rules of acceptance. Also, it makes it possible to vary safely the treatment parameters while helping the health care providers and while limiting the risks and the range of potential errors.

Of course, the variant embodiments according to the invention comprise the various known extracorporeal purification circuit configurations but also the possibility of using different solutes comprising citrate and calcium and injecting them at different locations of the circuit. For example, the pre-dilution solution contained in the bag 10′ can contain the citrate used as anticoagulant, or the dialyzate contained in the bag 9′ can contain the calcium used to restore the calcium level of the patient. As another example, the solute containing citrate can use any formula of sodium citrate or of citrate-dextrose (ACD), whereas calcium is injected in the concentrated form of calcium chloride or calcium gluconate or with a physiological concentration contained in the dialyzate or the substitution liquid. The concentrations of citrate and calcium can obviously vary from one fluid to the next and therefore will have to be entered by way of the user interface 21 so that the masses that are injected and rejected from the circulation loop 1, 2, 3 can be evaluated by the calculating unit 22. Other electrolytes can also be considered in the model, particularly sodium injected with citrate in the case of using sodium citrate or chlorine mixed with the calcium solution. Actually, a hypernatremia, resulting from a significant injection of sodium, can cause serious brain lesions by tearing the meningeal vessels, whereas chlorine is itself a major element affecting the determination of the pH. Also, the presence of magnesium in the solutes injected into the circulation loop 1, 2, 3 can be taken into consideration since magnesium binds to citrate and reduces accordingly the capacity of the latter to bind to calcium.

1-10. (canceled)

11. Device for purifying the blood of a patient comprising: an extracorporeal blood circulation loop, flow rate control means to manage the circulation of the blood in the extracorporeal blood circulation loop, blood purification means configured on the extracorporeal blood circulation loop, citrate injection means configured to inject a solution comprising citrate into the extracorporeal blood circulation loop, and a control unit configured to control said citrate injection means, and said flow rate control means , wherein the control unit is further configured to require at determined intervals of time the entry of at least one representative value of the status of the device and/or of the patient, to verify the adequacy of said representative value with pre-established rules stored in the control unit, and to control said citrate injection means and/or the flow rate control means in accordance with the result of said verification and said pre-established rules.

12. The device according to claim 11, wherein said representative value is selected from among the following values taken alone or in combination: the ionized calcium level of the patient undergoing treatment by the device, the ionized calcium/total calcium ratio of said patient, the ionized calcium level taken at a point of the extracorporeal blood circulation loop, the pH of said patient.

13. The device according to claim 11, further comprising pre-dilution means configured upstream from the blood purification means for injecting a pre-dilution liquid into the blood circulation loop, the control unit also being configured to control said pre-dilution means in accordance with the result of said verification and said pre-established rules.

14. The device according to claim 11, further comprising post-dilution means configured downstream from the purification means for injecting a post-dilution liquid into the blood circulation loop, with the control unit also being configured to control said post-dilution means in accordance with the result of said verification and said pre-established rules.

15. The device according to claim 11, further comprising removal means that for removing a contaminated solution downstream from the purification means, with the control unit also being configured to control said removal means in accordance with said verification and said pre-established rules.

16. The device according to claim 11, further comprising dialyzate injection means for injecting a dialyzate into the extracorporeal blood circulation loop, with the control unit also being configured to control said dialyzate injection means in accordance with the result of said verification and said pre-established rules.

17. The device according to claim 11, further comprising calcium and/or magnesium injection means for injecting a solution comprising calcium and/or magnesium into the extracorporeal blood circulation loop, downstream from the purification means if they are present, and wherein the control unit is also configured to control said calcium and/or magnesium injection means in accordance with the result of said verification and said pre-established rules.

18. The device according to claim 11, wherein at least one of the pre-established rules stored in the control unit prevents the citrate injection means from injecting into the blood circulation loop an amount of citrate that is greater than a first limit value, corresponding to the tolerance threshold of the patient.

19. The device according to claim 11, wherein at least one of the pre-established rules stored in the control unit prevents the citrate injection means from injecting into the blood circulation loop an amount of citrate that is smaller than a second limit value, corresponding to the threshold below which there exists a risk of coagulation of the blood in the extracorporeal blood circulation loop.

20. The device according to claim 11, wherein the control unit is further configured to reevaluate at each verification the period of the time intervals determined between each request for entry of a representative value.

21. The device according to claim 13, further comprising post-dilution means configured downstream from the purification means for injecting a post-dilution liquid into the blood circulation loop, with the control unit also being configured to control said post-dilution means in accordance with the result of said verification and said pre-established rules.

22. The device according to claim 13, further comprising removal means that for removing a contaminated solution downstream from the purification means, with the control unit also being configured to control said removal means in accordance with said verification and said pre-established rules.

23. The device according to claim 13, further comprising dialyzate injection means for injecting a dialyzate into the extracorporeal blood circulation loop, with the control unit also being configured to control said dialyzate injection means in accordance with the result of said verification and said pre-established rules.

24. The device according to claim 13, further comprising calcium and/or magnesium injection means for injecting a solution comprising calcium and/or magnesium into the extracorporeal blood circulation loop, downstream from the purification means if they are present, and wherein the control unit is also configured to control said calcium and/or magnesium injection means in accordance with the result of said verification and said pre-established rules.
